(19) United States
(12) Patent Application Publication
(10) Pub. No.: US 2013/0084241 A1
(43) Pub. Date: Apr. 4, 2013

(54) DEVELOPMENT OF miRNA DIAGNOSTICS TOOLS IN BLADDER CANCER

(71) Applicant: Board of Regents, The University of Texas System, Austin, TX (US)

(72) Inventors: Liana ADAM, Pearland, TX (US); Colin P. DINNEY, Houston, TX (US); David B. JACKSON, Heidelberg (DE)

(73) Assignee: BOARD OF REGENTS, THE UNIVERSITY OF TEXAS SYSTEM, Austin, TX (US)

(21) Appl. No.: 13/629,145

(22) Filed: Sep. 27, 2012

Related U.S. Application Data
(60) Provisional application No. 61/539,627, filed on Sep. 27, 2011.

(51) Int. Cl. C12Q 1/68

(52) U.S. Cl. CPC C12Q 1/6876
USPC 424/1.11; 506/9; 506/16; 514/44 R; 514/9.7; 514/1.1

(57) ABSTRACT

The present invention includes methods and compositions related to diagnosis of bladder cancer, including the presence of bladder cancer and/or the type or stage of bladder cancer. In specific embodiments, the expression of one, two, three, four, five, or more miRNAs of the invention are associated with detection of bladder cancer, typing of bladder cancer, or staging of bladder cancer. Kits and microarrays are encompassed in the invention.

This application claims the benefit of U.S. Provisional Patent Application No. 61/539,627, filed Sep. 27, 2011, the entirety of which is incorporated herein by reference.

### STATEMENT REGARDING FEDERALLY SPONSORED RESEARCH OR DEVELOPMENT

This invention was made with government support under a Specialized Program of Research Excellence (SPORE) grant P50 CA091846 funded by the National Cancer Institute. The government has certain rights in the invention.

### TECHNICAL FIELD

The field of the invention includes at least molecular biology, cell biology, and medicine, including cancer diagnostics.

### BACKGROUND OF THE INVENTION

Bladder cancer (BC) follows a so-called “dual-track” carcinogenesis concept that was developed three decades ago based on clinicopathologic observations (Dinney et al., 2004; Wu, 2005). Most BCs are papillary (i.e., “superficial”) lesions that almost always recur and sometimes evolve into higher-grade, invasive cancers. Patients with papillary BC must undergo regular surveillance for recurrence and consequential surgeries, thus making it the most expensive tumor type in terms of clinical management. In contrast, about 20% of BCs are nonpapillary and invasive at diagnosis. These tumors arise from severe dysplasia or carcinoma in situ (CIS). CIS indicates a dangerous process of tumor development and a high propensity for progression to invasive disease. Nonpapillary tumors account for the bulk of BC-related mortality, which amounts to 14,689 deaths per year in the United States, roughly 19% of the annual incidence of 70,530 BCs (2010, NCI statistics).

Developing novel blood and urine markers for the informative and noninvasive screening, detection, and surveillance of BC is vitally important for managing this disease, and identifying these markers is a top priority for the National Cancer Institute. Several markers have been approved by the U.S. Food and Drug Administration for the detection or surveillance of BC, but all have limitations that minimize their utility. The sensitivity of all the currently approved markers is too low to render them comparable to cystoscopy for detection, and their modest specificity and positive predictive value make urologists hesitant to initiate treatment on the basis of the results. MiRNAs have been suggested as promising biomarkers for detecting cancer, predicting prognosis, and assessing treatment response and as targets for prevention and therapy. From a biological standpoint, miRNAs are better predictive markers than messenger RNAs (mRNAs), because a single miRNA may regulate hundreds of mRNAs that are usually grouped in biological pathways; therefore, a more focused miRNA signature may provide as much information as several orders of magnitude more mRNAs (Cahn and Croce, 2006; Bartel, 2009). From a practical viewpoint, miRNAs are also more stable than mRNAs or proteins and less subject to degradation during sample processing; thus, miRNAs are more suitable for analysis in formalin-fixed paraffin-embedded tissues, urine, serum, or plasma (Bartel, 2009; Cortez and Calin, 2009).

### BRIEF SUMMARY OF THE INVENTION

The present invention is directed to methods and compositions related to cancer diagnosis and/or prognosis. In specific embodiments, the cancer is bladder cancer (BC), although in some embodiments the cancer is lung, brain, pancreatic, prostate, breast, colon, ovarian, spleen, esophageal, stomach, gall bladder, thyroid, rectum, ovarian, testicular, kidney, bone, blood, or skin cancer.

In embodiments of the invention, the present invention is applicable for any type of bladder cancer, including transitional cell carcinomas, squamous cell carcinoma and/or adenocarcinoma. The present invention is applicable to bladder cancer as a primary cancer and/or as metastatic cancer, in particular embodiments. In certain aspects, the present invention is useful for identifying bladder cancer and/or identifying muscle-invasive bladder cancer or non-muscle-invasive bladder cancer. In some embodiments the invention is employed for a human mammal, although other mammals are encompassed, such as dogs, cats, horses, and so forth.

In specific embodiments, the present invention provides assessment of the expression of known and predicted non-coding RNA species in the blood (for example) of individuals with or without BC and identification of disease-associated systemic miRNA footprints useful for diagnostic screening.

Exemplary miRNAs useful for diagnosing bladder cancer in an individual or a type or stage thereof are hsa-miR-1246; hsa-miR-33b; hsa-miR-1290; hsa-miR-92b*-AS; hsa-miR-923-P; hsa-miR-1826; hsa-miR-92b; hsa-miR-1268-AS; hsa-miR-923; hsa-miR-337-5p-AS, or a combination thereof of 2, 3, 4, 5, 6, 7, 8, 9 or all of them.

In some embodiments, exemplary miRNAs useful for diagnosing bladder cancer in an individual or a type or stage thereof include hsa-miR-92b; hsa-miR-1826; hsa-miR-92b*-AS; hsa-miR-33b; hsa-miR-1246; hsa-miR-1290; hsa-miR-1268-AS; hsa-miR-1914; hsa-miR-923-P; hs a-miR-23a; hsa-miR-923; hsa-miR-1469-AS; hsa-miR-184-P; hsa-miR-219-1-3p; hsa-miR-25; hsa-miR-935; hsa-miR-23b; hsa-miR-92a; hsa-miR-1228*-AS; hsa-miR-520c-3p-AS; hsa-miR-566-P; hsa-miR-33a-AS; hsa-miR-1254; hsa-miR-1181; hsa-miR-155*MM1T/C; hsa-miR-487a; hsa-miR-1273; hsa-miR-541; hsa-miR-195*; hsa-miR-487b; hsa-miR-148b; hsa-miR-634; hsa-miR-155MM1G/A; hsa-miR-1197; hsa-miR-548h; hsa-miR-32; hsa-miR-720; hsa-miR-202-AS; hsa-miR-937-AS, or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, or all of them. The miRNAs may be employed for distinguishing an individual with bladder cancer or not. In specific embodiments, low expression of those listed therein except hsa-miR-520c-3-AS, hsa-miR-566-P; hsa-miR-33a-AS; hsa-miR-1254; hsa-miR-487a; hsa-miR-1273; hsa-miR-541; hsa-miR-487b; hsa-miR-148b; and hsa-miR-634 are indicative of cancer, and hsa-miR-520c-3-AS, hsa-miR-566-P; hsa-miR-33a-AS; hsa-miR-1254; hsa-miR-487a; hsa-miR-1273; hsa-miR-541; hsa-miR-487b; hsa-miR-148b; and/or hsa-miR-634 having high expression are indicative of cancer. Thus, in some aspects, a method comprises (a) identifying the individual as having a bladder cancer if the expression level of one or more of hsa-miR-520c-3p-AS, hsa-miR-566-P; hsa-miR-33a-AS; hsa-miR-1254; hsa-miR-487a; hsa-miR-1273; hsa-miR-541; hsa-miR-487b; hsa-miR-148b; or hsa-miR-634 is increased in the sample relative to a reference or if the expression level of one or more of hsa-miR-92b; hsa-miR-1826; hsa-miR-92b*-AS; hsa-miR-33b; hsa-miR-1246; hsa-miR-1290; hsa-miR-1268-AS; hsa-miR-1914; hsa-miR-923-P; hsa-miR-23a; hsa-miR-923; hsa-miR-1469-AS; hsa-miR-184-P; hsa-miR-219-1-3p; hsa-miR-25; hsa-miR-935; hsa-miR-23b; hsa-miR-92a; hsa-miR-1228*-AS; hsa-miR-1181; hsa-miR-155*MM1T/C; hsa-miR-195*; hsa-miR-155MM1G/A; hsa-miR-1197; hsa-miR-548h; hsa-miR-32; hsa-miR-720; hsa-miR-202-AS or hsa-miR-937-AS is decreased in the sample relative to a reference; or (b) identifying the individual as not having a bladder cancer if the expression level of hsa-miR-520c-3p-AS, hsa-miR-566-P; hsa-miR-33a-AS; hsa-miR-1254; hsa-miR-487a; hsa-miR-1273; hsa-miR-541; hsa-miR-487b; hsa-miR-148b; or hsa-miR-634 is not increased in the sample relative to a reference or if the expression level of hsa-miR-92b; hsa-miR-1826; hsa-miR-92b*-AS; hsa-miR-33b; hsa-miR-1246; hsa-miR-1290; hsa-miR-1268-AS; hsa-miR-1914; hsa-miR-923-P; hsa-miR-23a; hsa-miR-923; hsa-miR-1469-AS; hsa-miR-184-P; hsa-miR-219-1-3p; hsa-miR-25; hsa-miR-935; hsa-miR-23b; hsa-miR-92a; hsa-miR-1228*-AS; hsa-miR-1181; hsa-miR-155*MM1T/C; hsa-miR-195*; hsa-miR-155MM1G/A; hsa-miR-1197; hsa-miR-548h; hsa-miR-32; hsa-miR-720; hsa-miR-202-AS or hsa-miR-937-AS is not decreased in the sample relative to a reference.

In some embodiments, exemplary miRNAs useful for diagnosing bladder cancer in an individual or a type or stage thereof include hsa-miR-1826; hsa-miR-604; hsa-miR-1246; hsa-miR-33b; hsa-miR-92b; hsa-miR-1290; hsa-miR-92a; hsa-miR-1268-AS; hsa-miR-1914; hsa-miR-92b*-AS; hsa-miR-940-P; hsa-miR-181a-2*-AS; hsa-miR-423-3p; hsa-miR-219-1-3p; hsa-miR-25; hsa-miR-541; hsa-miR-522-AS; hsa-miR-574-3p; hsa-miR-184-P; hsa-miR-1263-P; hsa-miR-1250-P; hsa-miR-302b; hsa-miR-338-3p-AS; hsa-miR-212; hsa-miR-200b; hsa-miR-373*-AS; hsa-miR-671-3p; hsa-miR-1255b; hsa-miR-1262; hsa-miR-553; hsa-miR-544; hsa-miR-923-P; hsa-miR-1248-P; hsa-miR-1233; hsa-miR-923; hsa-miR-494; hsa-miR-1469-AS; hsa-miR-520c-3p-AS; hsa-miR-23b; hsa-miR-520d-3p-AS, or a combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or all of them. The miRNAs may be employed for distinguishing an individual with invasive bladder cancer or not. In some embodiments, high expression of one or more of these is indicative of invasive bladder cancer, wherein in some embodiments low expression of one or more of these is indicative of invasive bladder cancer. Examples of those having high expression being indicative of invasive cancer include hsa-miR-604; hsa-miR-940-P; hsa-miR-181a-2*-AS; hsa-miR-423-3p; hsa-miR-541; hsa-miR-522-AS; hsa-miR-574-3p; hsa-miR-1263-P; hsa-miR-338-3p-AS; hsa-miR-212; hsa-miR-200b; hsa-miR-671-3p; hsa-miR-1255p; hsa-miR-1262; hsa-miR-553; hsa-miR-544; hsa-miR-1248-P; hsa-miR-1233; hsa-miR-520c-3p-AS; and/or hsa-miR-520d-3p-AS. Examples of those having low expression being indicative of invasive cancer include hsa-miR-1826; hsa-miR-1246; hsa-miR-33b; hsa-miR-92b; hsa-miR-1290; hsa-miR-92a; hsa-miR-1268-AS; hsa-miR-1914; hsa-miR-92b*-AS; hsa-miR-219-1-3p; hsa-miR-25; hsa-miR-184-P; hsa-miR-1250-P; hsa-miR-302b; hsa-miR-373*-AS; hsa-miR-923-P; hsa-miR-923; hsa-miR-494; hsa-miR-1469-AS; and/or hsa-miR-23b. Thus, in further aspects, a method of the embodiments comprises (a) identifying the individual as having an invasive bladder cancer if the expression level of one or more of hsa-miR-604; hsa-miR-940-P; hsa-miR-181a-2*-AS; hsa-miR-423-3p; hsa-miR-541; hsa-miR-522-AS; hsa-miR-574-3p; hsa-miR-1263-P; hsa-miR-338-3p-AS; hsa-miR-212; hsa-miR-200b; hsa-miR-671-3p; hsa-miR-1255p; hsa-miR-1262; hsa-miR-553; hsa-miR-544; hsa-miR-1248-P; hsa-miR-1233; hsa-miR-520c-3p-AS; or hsa-miR-520d-3p-AS is increased in the sample relative to a reference or if the expression level of one or more of hsa-miR-1826; hsa-miR-1246; hsa-miR-33b; hsa-miR-92b; hsa-miR-1290; hsa-miR-92a; hsa-miR-1268-AS; hsa-miR-1914; hsa-miR-92b*-AS; hsa-miR-219-1-3p; hsa-miR-25; hsa-miR-184-P; hsa-miR-1250-P; hsa-miR-302b; hsa-miR-373*-AS; hsa-miR-923-P; hsa-miR-923; hsa-miR-494; hsa-miR-1469-AS; or hsa-miR-23 is decreased in the sample relative to a reference; or (b) identifying the individual as not having an invasive bladder cancer if the expression level of hsa-miR-604; hsa-miR-940-P; hsa-miR-181a-2*-AS; hsa-miR-423-3p; hsa-miR-541; hsa-miR-522-AS; hsa-miR-574-3p; hsa-miR-1263-P; hsa-miR-338-3p-AS; hsa-miR-212; hsa-miR-200b; hsa-miR-671-3p; hsa-miR-1255p; hsa-miR-1262; hsa-miR-553; hsa-miR-544; hsa-miR-1248-P; hsa-miR-1233; hsa-miR-520c-3p-AS; or hsa-miR-520d-3p-AS is not increased in the sample relative to a reference or if the expression level of hsa-miR-1826; hsa-miR-1246; hsa-miR-33b; hsa-miR-92b; hsa-miR-1290; hsa-miR-92a; hsa-miR-1268-AS; hsa-miR-1914; hsa-miR-92b*-AS; hsa-miR-219-1-3p; hsa-miR-25; hsa-miR-184-P; hsa-miR-1250-P; hsa-miR-302b; hsa-miR-373*-AS; hsa-miR-923-P; hsa-miR-923; hsa-miR-494; hsa-miR-1469-AS; or hsa-miR-23 is not decreased in the sample relative to a reference.

Labels can be attached to miRNA including those that are covalently attached to a nucleic acid. It is contemplated that the label on labeled nucleotides or the label that becomes attached to the nucleotides in a miRNA is biotin, radioactivity, or a dye. Alternatively, the label may be qualified as positron-emitting, colorimetric, enzymatic, luminescent, fluorescent, or a ligand.

In some embodiments, methods involve identifying an appropriate sample to analyze or evaluate. It is particularly contemplated that in some embodiments, an appropriate sample is one that can provide information about a particular disease or condition or about some other phenotype. Other methods of the invention concern analyzing miRNA in a sample comprising generating an miRNA profile for the sample and evaluating the miRNA profile to determine whether miRNA in the sample are differentially expressed compared to a normal sample. In specific embodiments, methods of the invention include a method for evaluating miRNA in a biological sample. In certain instances, the biological sample is from a patient. This method is implemented by analyzing one or more miRNAs in a sample using the array compositions and methods of the invention. In specific embodiments, miRNA are evaluated by one or more of the following steps: a) isolating miRNA away from other RNA in the sample; b) labeling the miRNA; c) hybridizing the miRNA to an miRNA array; and, d) determining miRNA hybridization to the array. Whether miRNAs hybridize to the array, what miRNAs hybridize to the array, and/or how much total miRNA or any specific miRNAs hybridize to the array are ways of determining the extent of miRNA hybridization to the array. Methods of detecting, measuring and quantifying hybridization are well known to those of skill in the art. In specific embodiments, miRNA hybridization is quantified.

The present invention also concerns methods of generating a miRNA profile for a sample. The term “miRNA profile” refers to a set of data regarding the expression pattern for a plurality of miRNAs in the sample that was obtained using a miRNA array. ill some embodiments of the invention, an miRNA profile is generated by steps that include: a) labeling miRNA in the sample; b) hybridizing the miRNA to a miRNA array; and, c) determining miRNA hybridization to the array, wherein a miRNA profile is generated. miRNA profiles can be generated to compare differences in miRNA expression between any two or more different samples. miRNA profiles can be compared, for example, between a sample with a particular disease, disorder, or condition and a sample that does not have the particular disease, disorder or condition; between samples that have a particular disease, disorder or condition but a different stage of the disease, disorder or condition; between samples that have a particular disease, disorder or condition but with a different prognosis with respect the disease, disorder or condition; between a sample that has been treated with a particular agent and a sample that has not been treated with that agent; between samples that have responded differently to a particular substance or agent, such as one responsive to the treatment and one not, or one resistant to the treatment and one not; samples that differ by gender of the sources; samples that differ by age or stage of development of the source; samples that differ by tissue type; samples that differ by at least one known polymorphism; between a sample that has a particular mutation and a sample that does not; a sample that is defective in a particular pathway or has a defective protein and a sample that does not; between a sample that is apparently resistant to a particular disease, disorder, or condition and a sample that is not expected to be resistant to that particular disease, disorder, or condition, as well as a comparison involving any samples with a combination of characteristics as described above.

Samples from which miRNA profiles are generated include samples that can be characterized based on one or more of the following: age; developmental stage; prognosis of a disease, condition, or disorder; cell type; tissue type; organ type; race or ethnicity; gender; susceptibility to or risk of a particular disease, condition, or disorder; diet; exposure to or treatment with a particular chemical, agent. or substance; diagnosis of a particular a disease, condition, or disorder; organism type; genomic makeup, etc.

Methods of the invention allow differences between two or more biological samples to be determined by generating an miRNA profile for each sample and comparing the profiles, wherein a difference in the profiles identifies differentially expressed miRNA molecules. In specific embodiments, a first sample is treated with a substance prior to generating the miRNA profile and a second sample is untreated. In other embodiments, a first sample exhibits a disease or condition and a second sample exhibits the same disease or condition but at a different stage of progression. In further embodiments, a first sample responds favorably to a therapeutic agent and a second sample is unresponsive to the therapeutic agent. Moreover, in other embodiments, a first sample is from a first subject who responds adversely to a therapeutic agent and a second sample is from a second subject does not respond adversely to the therapeutic agent.

Other methods of the invention concern identifying a correlation between miRNA expression and a disease or condition comprising comparing different miRNA profiles, such as 1) an miRNA profile of a sample with the disease or condition or from a subject with the disease or condition and 2) an miRNA profile of a sample that is normal with respect to that disease or condition or that is from a subject that does not have the disease or condition. In specific embodiments, methods include a) isolating miRNA from a sample exhibiting the disease or condition; b) labeling the miRNA; c) hybridizing the miRNA to an miRNA array; and, d) identifying miRNA differentially expressed in the sample compared to a normal sample. It is contemplated that the miRNA profiles may be generated in the process of performing the method; alternatively, they may be obtained from previously obtained results. Moreover, it is contemplated that comparisons may be done by using a plurality of miRNA profiles (multiple samples from the same source obtained at the same or different times and/or samples from different sources). In this case, a normalized miRNA profile may be generated and used for comparison purposes.

In certain embodiments, methods concern identifying miRNAs indicative of a disease or condition by detecting a correlation between the expression of particular miRNAs and a sample believed to have a disease or condition. In further aspects, method concern identifying individuals having bladder cancer or invasive bladder cancer. In certain aspects, a step of “identifying” comprises reporting miRNA expression levels in a sample or reporting whether an individual has an bladder cancer or an invasive (e.g., muscle invasive) bladder cancer. For example, a reporting can comprise providing a written, electronic or oral report. In some cases a report is provided to an individual (e.g., a patient), a health care worker, a hospital or an insurance company.

In specific embodiments, there are methods for analyzing a biological sample from a patient for a disease or condition comprising generating an miRNA profile for the sample and evaluating the miRNA profile to determine whether miRNA in the sample are differentially expressed compared to a normal sample. The comparison may involve using an array that has selective miRNA probes that are indicative of a disease or condition. Arrays of the invention include macroarrays and microarrays.

Cancer includes, but is not limited to, malignant cancers, tumors, metastatic cancers, unresectable cancers, chemo- and/or radiation-resistant cancers, and terminal cancers. It is specifically contemplated that in any embodiments involving a possible decrease or increase in expression of certain miRNAs that only a decrease may be evaluated, only an increase may be evaluated, or that both an increase and decrease in expression of any of the miRNA mentioned in that context (or any other discussed herein) may be evaluated. Accordingly, in a further embodiment there is provided a method of treating an individual diagnosed with a bladder cancer (e.g., an invasive bladder cancer, such as a muscle invasive bladder cancer) by a method of the embodiments comprising administering an anticancer therapy to the individual. For example, the anticancer therapy can be a chemotherapy, radiotherapy, gene therapy, surgery, hormonal therapy, anti-angiogenic therapy or cytokine therapy.

Throughout this application, the term “difference in expression” or analogous language thereof means that the level of a particular miRNA in a sample is higher or lower than the level of that particular miRNA in a normal sample. “Normal sample” in the context of testing for cancer means a noncancerous sample.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1. Principle component analysis and hierarchical clustering of samples based on expression levels of all 9600 assayed sequences. The expression profiling of human miRNAs represented by (A) the first two principle components or by (B) unsupervised clustering cannot clearly distinguish the BC from control samples.

FIG. 2. Differentially expressed miRNAs correlate with various disease states. (A) miR-1290 and (B) miR-92b expression correlation with pathological grade and invasiveness; (C) clustering of samples using discriminative miRNAs reflects histological grade and disease state.

FIG. 3. Logistic regression (LR) analysis results. (A) Correlation plot between each miRNA duplicate value. (B) LOO-ROC curve for LR classifier for cancerous (MIBC or NMIBC) vs non-cancerous or (C) MIBC vs other (NMIBC or non-cancerous). The red circle corresponds to the natural probability threshold of 0.5. (C), LOO-ROC curve for LR classifier for MIBC vs controls. Dotted line, random prediction.

FIG. 4. The 40 most important features as determined from trained classifiers. (A), cancerous vs non-cancerous; (B), MIBC vs NMIBC/controls.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein. Embodiments discussed in the context of methods and/or compositions of the invention may be employed with respect to any other method or composition described herein. Thus, an embodiment pertaining to one method or composition may be applied to other methods and compositions of the invention as well.

In embodiments of the invention, there is systemic microRNA measurement as a useful tool for predicting diagnosis in cancer, including at least bladder cancer and, in specific embodiments, muscle-invasive bladder cancer.

The present invention provides novel urine markers for informative and non-invasive screening, detection, and surveillance of bladder cancer (BC). Somatic alterations of miRNAs have been suggested as promising biomarkers for early detection, prognosis and treatment response, and targets for prevention and therapy. In embodiments of the invention, tumor-host interactions during bladder carcinogenesis are reflected by variations in miRNA expression. These changes reflect a modification of miRNA homeostasis or identify cancer-prone homeostasis, during which the cellular interactions between tumor and other cells is modified, in certain embodiments of the invention. Specific embodiments of the invention provide a useful, noninvasive tool for clinically assessing BC with immediate applicability to patient care.

miRNA Arrays

The present invention also concerns arrays for evaluating miRNA molecules. Clearly contemplated is an array that is a microarray. The arrays have one or more probes directed to one or more miRNA molecules (“miRNA array”). In some embodiments, an miRNA array includes one or more miRNA probes immobilized on a solid support. An “miRNA probe” refers to a nucleic acid having a sequence that is complementary or identical to all or part of a miRNA precursor or gene such that it is capable of specifically hybridizing to an miRNA gene, the cognate miRNA precursor, or the processed miRNA. Typically, the probe will contain at least ten contiguous nucleotides complementary to all or part of the miRNA precursor or at least ten contiguous nucleotides complementary or identical to all or part of 30 an miRNA gene. It will be understood that DNA probes with sequences relative to an miRNA gene will be identical in sequence to all or part of the coding sequence of the gene and complementary in sequence to all or part of the noncoding sequence of the gene. In specific embodiments, an miRNA probe contains the sequence encoding an miRNA (“miRNA coding sequence,” which refers to sequence encoding processed miRNA). Because the precise length and, consequently, sequence of a particular processed miRNA has been found to vary occasionally, the predominant species will be understood as the sequence and length of the processed miRNA. The predominant species is usually the one observed at least 90% of the time.

The number of different probes on the array is variable. It is contemplated that there may be, be at least, or be at most 1, 2, 3, 4,5,6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more, or any range derivable therein, different miRNA probes on an array. ill specific embodiments, arrays have between 5 and 1000 different miRNA probes, between 20 and 500 different miRNA probes, between 50 and 250 different miRNA probes, or between 100 and 225 different miRNA probes. “Different” probes refers to probes with different sequences. Therefore, it is. contemplated that different probes can be used to target the same miRNA. Moreover, multiple and different probes to the same miRNA can be included on an array. For example, one probe may target specifically a precursor miRNA or the miRNA gene (depending on what sample is used to hybridize to the array—i.e, whether the sample contains DNA or RNA), while another probe may be capable of hybridizing to the processed miRNA, its precursor, or the gene.

Moreover, miRNA probes targeting the same miRNA may be overlapping, such that they share contiguous sequences. It is also contemplated that a single probe may target multiple miRNAs, particularly miRNAs from the same gene family or related miRNAs (distinguished by a letter). It is understood by those of skill in the art that a “gene family” refers to a group of genes having the same miRNA coding sequence. Typically, members of a gene family are identified by a number following the initial designation. For example, miR-16-1 and miR-16-2 are members of the miR-16 gene family. Also, a probe may have a sequence that allows it to target more than 1 miRNA. It is understood that a 2-base 30 pair mismatch between the probe and an miRNA is sufficient to hybridize with at least 90% of the mismatched miRNA under the conditions described in the Examples. Consequently, it will be understood that unless otherwise indicated, a probe for a particular miRNA will also pick up a related miRNA, such as those designated with the same number but with an added letter designation. For example, an miRNA probe that is fully complementary to miR-15a would also hybridize to miR-15b, unless otherwise noted. Thus, an miRNA probe can target 1, 2, 3, 4, 5, 6 or more different miRNAs. miRNA probes are contemplated to be made of DNA, though in some embodiments, they may be RNA, nucleotide analogs, PNAs, or any combination of DNA, RNA, nucleotide analogs, and PNAs.

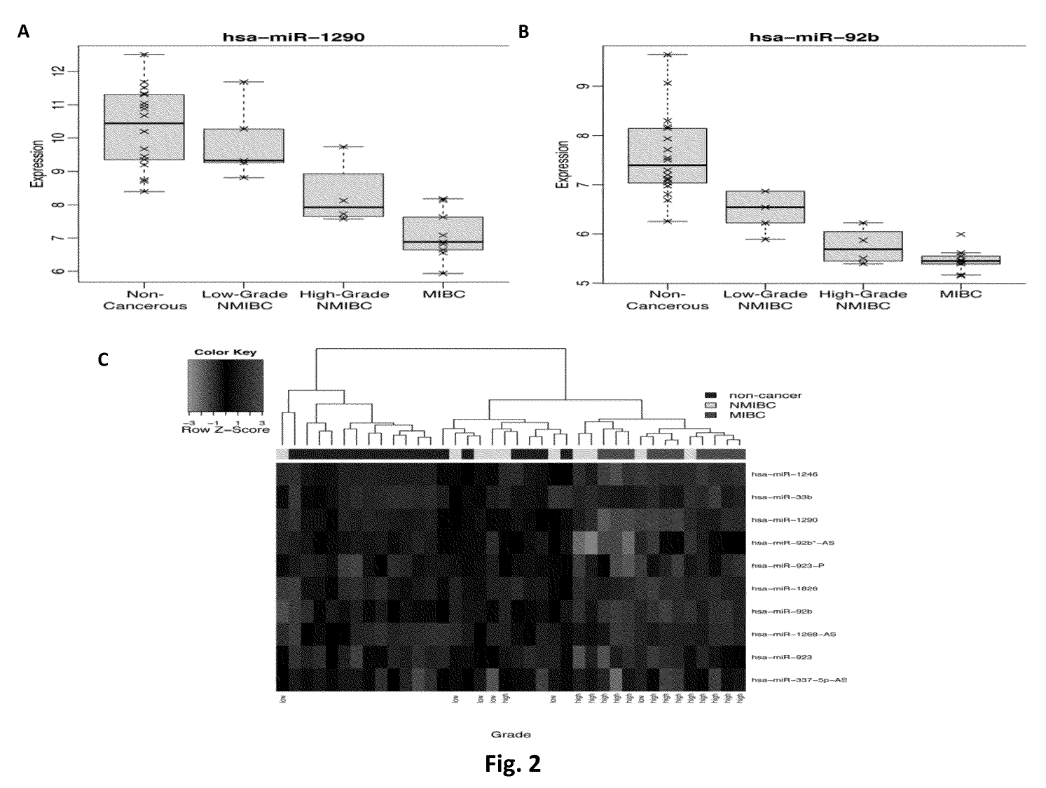

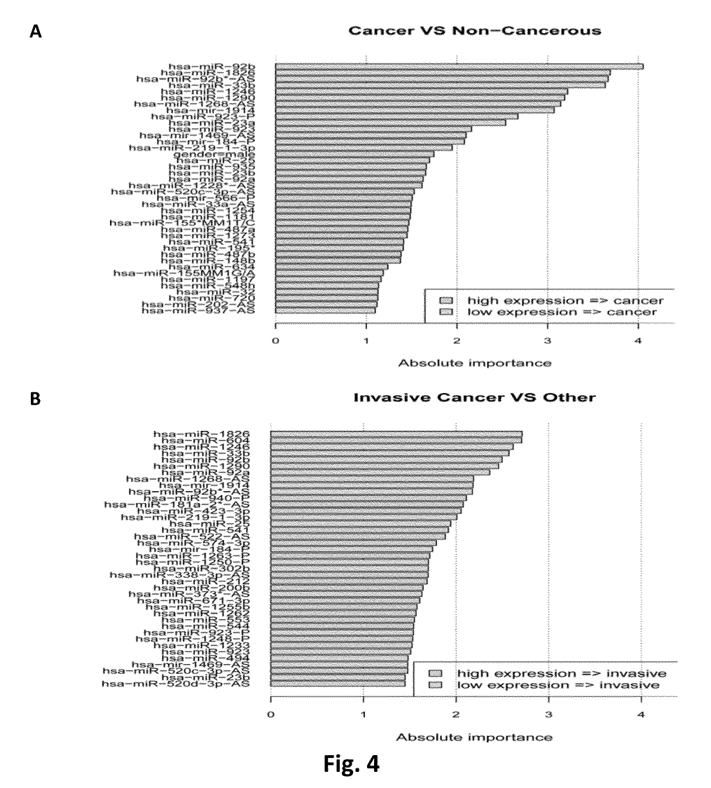

miRNA probes of the invention have an miRNA coding sequence that is between 19-34 nucleotides in length. Of course, this is understood to mean that the probes have 19-34 contiguous nucleotides that are identical or nearly identical to the miRNA gene and complementary to the processed miRNA or its precursor. As discussed above, a probe can be used to target an miRNA with which it has a 2-base pair mismatch in hybridization. Thus, it is contemplated that miRNA probes of the invention may be almost fully complementary (2 base-pair mismatches or fewer) or fully complementary to any miRNA sequence or set of sequences (such as related miRNAs or miRNAs from the same gene family) that is targeted. The term “nearly identical” means that any difference in sequence is 2 bases or fewer. When an miRNA has a perfectly complementary stem loop in its precursor, the miRNA coding sequence should be identical to a sequence in the precursor as well. ill some embodiments of the invention, a probe has an miRNA coding sequence that includes the entire processed miRNA sequence. It is contemplated that the probe has or has at least 5, 6, 7, 8, 9, 10, 11, 12, 13, 14, 15, 16, 17, 18, 19, 20, 21, 22, 23, 24, 25 or more contiguous nucleotides, or any range derivable therein, from an miRNA coding sequence. In specific embodiments, an miRNA probe has a sequence identical or complementary, or at least 90% or greater identity or complementarity, across the lengths discussed in the previous sentence with respect to any of the miRNAs of FIG. 2 or 4.

As discussed above, miRNA are processed from a precursor molecule. In certain embodiments, probes have an miRNA coding sequence that also includes at least 2 to 5 nucleotides of coding sequence upstream and/or downstream of the processed miRNA sequence. Probes may have or have up to 1, 2, 3, 4, 5, 6, 7, or more contiguous nucleotides, or any range derivable therein, that flank the sequence encoding the predominant processed miRNA on one or both sides (5′ and/or 3′ end). ill particular embodiments, probes have an miRNA coding sequence that includes 4 nucleotides of coding sequence upstream (5′) and/or downstream (3′) of the processed miRNA sequence. On other embodiments, miRNA probes also have one or more linkers flanking the miRNA coding sequence. ill particular embodiments, there is a linker at the 3′ end of the miRNA coding sequence. ill some embodiments, a linker has a sequence that is between 3 to 25 nucleotides in length.

In some embodiments of the invention, miRNA probes are attached to the array through an amine attached at the 3′ end. The invention is not limited to arrays constructed with particular materials. Typically, arrays are made with materials that do not interfere with the hybridization between the probe and a sample. In some embodiments, the array is a solid support that is made with glass, plastic, or metal.

The present invention concerns methods for identifying a correlation between miRNA expression and a disease or condition. ill certain embodiments, methods involve identifying miRNA differentially expressed in a sample representative of the disease or condition (non-normal sample) compared to a normal sample. A sample representative of the disease or condition will be one that has the disease or condition, is affected by the disease or condition, and/or causes the disease or condition. In certain embodiments, identifying differentially expressed miRNA involves: a) labeling miRNA in the sample; and b) hybridizing the labelled miRNA to an miRNA array. ill further embodiments, the miRNA in the sample is isolated before or after labeling.

### Kits of the Invention

Any of the compositions described herein may be comprised in a kit. In a non-limiting example, one or more compositions for diagnosing, staging, or typing bladder cancer in one or more individuals may be comprised in a kit, and the composition(s) are comprised in a suitable container means. The compositions may include a substrate having one or more miRNAs of the present invention (for example, of FIG. 2 or FIG. 4) affixed thereto and/or may include part or all of the miRNA nucleic acids or nucleic acids that are complementary thereto and/or may include reagents useful to amplify (such as by polymerase chain reaction) miRNAs or hybridize miRNAs to a complementary sequence. A label and associated reagents for attaching a label to an entity such as nucleic acid may be included in the invention.

The kits may comprise a suitably aliquoted compositions of the present invention, where appropriate. The components of the kits may be packaged either in aqueous media or in lyophilized form, as necessary. The container means of the kits may generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the reagent containers in close confinement for commercial sale. Such containers may include injection or blow molded plastic containers into which the desired vials are retained. Some components of the kit may be provided as dried powder(s). When reagents and/or components are provided as a dry powder, the powder can be reconstituted by the addition of a suitable solvent. It is envisioned that the solvent may also be provided in another container means, in certain aspects.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Materials and Methods

### Patients and Sample Collection and Processing

The study associated with the present invention was approved by the Institutional Review Board at The University of Texas MD Anderson Cancer Center (LAB09-0149).

Whole blood samples were prospectively collected from patients with preoperative BC (n=20) or from control individuals without a known history of cancer of any type (n=18) (UMF-Timisoara). Over 70% were male with a median age of 55 in both groups. Each patient provided his/her informed consent according to IRB regulations implemented by MD Anderson Cancer Center and Municipal hospital-Timisoara, respectively. Total RNA was isolated from plasma and hybridized using custom-made noncoding RNA arrays (MD Anderson Cancer Center) which yielded 19,200 measurement values per patient (9,600 miRNAs, each in duplicate), as previously described (Liu et al., 2008). We defined all stage Ta-T1 disease, with or without CIS, as non-muscle-invasive BC (NMIBC), and we defined T2-T4 disease as muscle-invasive BC (MIBC). Grade was designated as low or high-grade.

### Reverse Transcriptase Polymerase Chain Reaction Analysis for Sample Quality Control Assessment

Reverse transcript polymerase chain reaction analysis was carried out using a TaqMan miRNA reverse transcription kit (Applied Biosystems) according to the manufacturer's instructions. We used 0.2 ng of total RNA for cDNA amplification using an arbitrarily primed multicolor detection system (Applied Biosystems). All assays were performed in triplicate, and miRNA expression levels were calculated using the comparative cycle threshold (Ct) method. The fold change was calculated using the equation 2-ΔΔCt.

### Statistical Analysis

All statistical analyses were performed in the statistics system R using bioconductor (Gentleman et al., 2004), further public packages, and custom programming.

The miRNA expression levels were normalized using a variance-stabilizing transformation (Huber et al., 2002). Many subsequent analyses implicitly rely on the variance being roughly constant over the range of expression levels, and quantile normalization failed to achieve this. For each miRNA, the Pearson correlation coefficient r of the duplicate measurements was computed to assess signal strength and reliability.

Clustering and principle component analysis were used for exploratory (i.e., “unsupervised”) analysis (Gehlenborg et al., 2010; Duda et al., 2001). We used t-tests and extensions thereof, specifically shrinkage t-tests (Opgen and Strimmer, 2007), followed by FDR control (Strimmer, 2008) to find differentially expressed miRNAs that correlated with the disease state, from NMIBC to MIBC. To develop systems for predicting diagnosis, we applied several machine learning methods (Duda et al., 2001): random forests of classification trees, nearest shrunken centroids, and regularized logistic regression (Zhu and Hastie, 2005). For each of the methods, we utilized an appropriate technique to estimate the generalization performance of the obtained classifiers, namely bootstrapping and leave-one-out cross-validation (LOO-CV). In a post-processing step, we extracted the importance of each miRNA to the resultant classifiers (Zien et al., 2009).

### Pathway Enrichment Analysis

To identify the miRNA signature that could discriminate tumor samples from normal samples or MIBC from normal samples, we performed a KEGG-based pathway enrichment analysis using DIANA-miRPath software for the gene targets predicted by DIANA microT Pic-Tar and TargetScan (see the Diana lab website at the Alexander Fleming Biomedical Sciences Research Center in Greece).

### Example 2

### Systemic MicroRNA Measurement for Diagnosis of Bladder Cancer

### Unsupervised Analysis

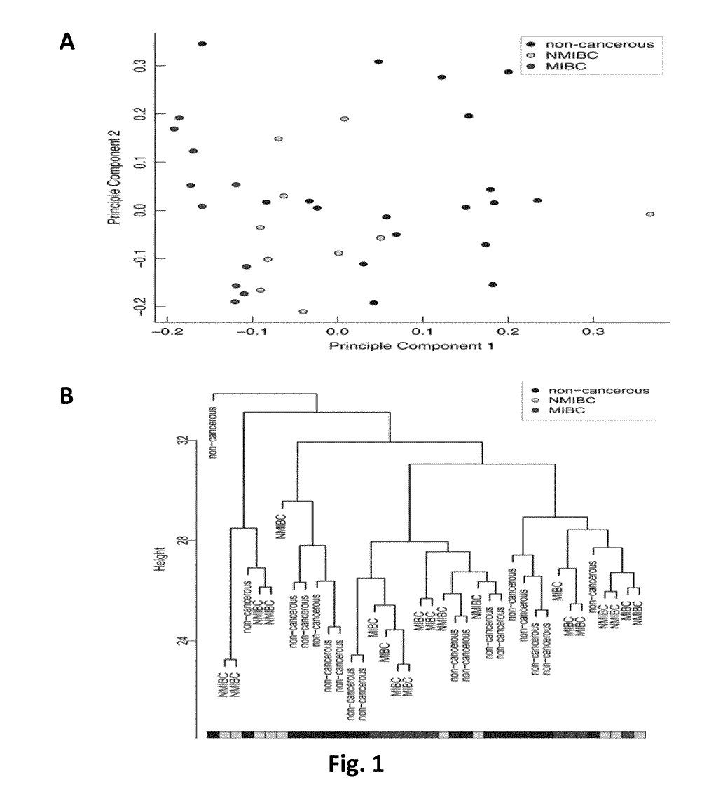

We first analyzed whether the miRNA expression levels could segregate the samples into two main groups, non-cancerous and BC. We used two standard approaches for exploratory data analysis, principle component analysis (FIG. 1A) and clustering (FIG. 1B), and subsequently verified whether the resultant grouping of the samples reproduced their known classification. The results of both approaches suggested that factors other than disease type and stage influence miRNA expression in the systemic blood circulation, and that some of those factors may dominate over the effect of BC. This was not an unexpected finding, since patients (usually of advanced age) both with and without BC had co-morbidities such as degenerative or metabolic diseases. Altogether, these findings suggest that a supervised analysis is necessary.

Clustering of Differentially Expressed microRNAs

We identified 10 miRNAs that are differentially expressed between BC and non-cancerous patients with high confidence (moderated t-test, tail-based FDR<10%). Several of the identified miRNAs, such as miR-1290 (FIG. 2A) and miR-92b (FIG. 2B), showed expression patterns that correlated well with an extended disease state. We re-clustered the samples using only these 10 miRNAs (FIG. 2C). Although MIBC samples showed a good separation from normal samples, the NMIBC samples showed an apparently wider distribution, one that overlapped with the invasive or normal distributions. We next reasoned that this distribution of the NMIBCs may be the reflection of other features (either clinical annotations or molecular signatures) that could further help separate the NMIBCs into additional subgroups. Indeed we found that the NMIBC cases that were previously identified as “normal” were mostly low grade, whereas almost all of the NMIBC cases previously identified as “MIBC” were high grade (FIG. 2C). However, when determining the diagnostic utility of a test one important caveat is that such supervised clustering may lead to overly optimistic estimates of classification accuracy, as it utilizes (via the miRNA selection) the known diagnoses of all patients and thus bears the danger of overfitting.

### Machine Learning Classification

Next, we identified 79 miRNAs that are likely to be systematically deregulated (local FDR<0.5) in the serum of patients with BC. None of these 79, however, suffices by itself to distinguish BC cases from control cases. Hence we assessed how accurately BC could be diagnosed from the measured miRNAs by machine learning methods that combine evidence from several miRNAs. We tried three classification methods: LR, nearest shrunken centroids, and random forests of classification trees (Table 1).

TABLE 1
Cross-validation accuracy for logistic regression.

| | | No. of | | | | |
|---|---|---|---|---|---|---|
| | No. of | CV | Accu- | Sensi- | Speci- | |
| | Cases | Errors* | racy* | tivity* | ficity* | auROC |
| Cancerous | 20 vs. 18 | 4 | 89% | 90% | 89% | 91% |
| vs Other | | | | | | |
| Invasive vs | 10 vs. 28 | 3 | 92% | 80% | 96% | 95% |
| Other | | | | | | |
| Invasive vs | 10 vs. 18 | 0 | 100% | 100% | 100% | 100% |
| Non- | | | | | | |
| Cancerous | | | | | | |

Measures marked with a star (*) correspond to binary predictions obtained by a 0.5 significance. auROC, area under the receiver operating characteristics curve.

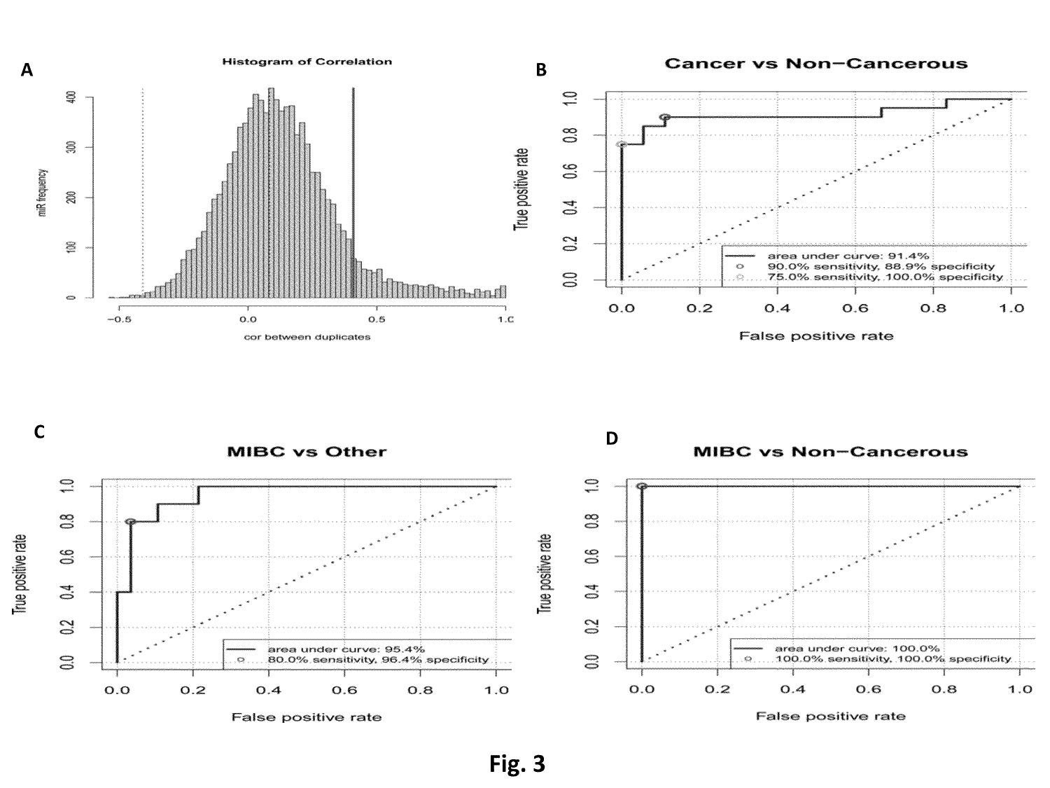

When training the classifiers, we faced the problem of determining many variables (e.g., the weight of each of the 9,600 candidate miRNAs) from far a small number of observations (<40 cases). To ease this task, we utilized the correlation coefficients “r” between the duplicate measurements (FIG. 3A). We excluded miRNAs with r<0.4 and weighted the expression data of the remaining miRNAs by multiplying them by r. This excludes silent miRNAs, because measurements dominated by noise are expected to yield low correlation coefficients. In addition to the retained miRNA expression levels, a binary indicator variable encoding patient gender was also used as a feature. The rationale for this is that the prevalence of BC is much higher in men than in women.

To obtain realistic estimates of predictive accuracy, we used bootstrapping and cross-validation. For instance, regularized LR was trained on all but one patient, and a prediction was made for the left-out patient (LOO-CV); the method cycled through all patients. The strength of the regularization was determined by maximizing the LOO negative log likelihood. An LR prediction was the estimated probability of the patient of being in one class (e.g., BC), given the miRNA measurements. A hard prediction was naturally derived by applying a threshold of 0.5. For cancer vs controls, this hard prediction yielded 90% sensitivity and 89% specificity (FIG. 3B, red circle). Changing the threshold can trade decreased sensitivity for increased specificity, or vice versa. For instance, applying a 0.8 threshold yielded the 75% sensitivity at 100% specificity, hence preventing any false alarm in the LOO-CV (FIG. 3B, orange circle). For MIBC vs others, we obtained 80% sensitivity and 96% specificity (FIG. 3C), whereas we could distinguish with 100% accuracy the MIBC cases from the controls (FIG. 3D).

Diagnostically Useful miRNAs

Last, we computed how much each miRNA contributed to the LR classifier. The 40 most diagnostically useful miRNAs were determined: first, for distinguishing BC from control samples (FIG. 4A); second, for MIBC versus other (NMIBC and normal) samples (FIG. 4B). Several miRNAs, such as miR-541, miR-200b, miR-566, miR-487, and miR-148b, were upregulated in the blood of patients with BC, whereas the expression of other miRNAs, such as miR-25, miR-92a, -92b, miR-302, and miR-33b, was significantly higher in control patients. These results indicate that a “footprint” of various miRNAs is associated with the onset of BC, in certain aspects of the invention.

### Significance of Certain Embodiments of the Invention

The results indicate the diagnostic potential of miRNA expression from the serum of patients with BC. LR was the most accurate statistical method for predicting diagnosis, with 89% accuracy for detecting the presence or absence of BC, 92% accuracy for distinguishing invasive BC from other cases, 79% accuracy for three-way classification, and 100% accuracy for distinguishing MIBC from control cases.

The value of miRNAs as biomarkers, specific to the tumor and/or the patient, has become apparent across a spectrum of cancers (Calin and Croce, 2006). These noncoding RNAs usually bind to mRNAs at their 3′ untranslated regions (UTRs), thereby triggering mRNA degradation or inhibition of protein translation (Liu et al., 2008). One miRNA can affect a multitude of genes depending on their sequence complementarities, and one gene can be affected by several miRNAs, which indicates a certain level of redundancy. Functional studies, however, have demonstrated that miRNAs have very specific targets, depending on the cellular types that express them. Furthermore, the miRNA function is usually grouped in a pathway manner, specific for the cellular type or tissue, in a more specific way than gene expression is, most likely owing to a reduction of the noise in miRNA expression patterns (Liu et al., 2008). How these miRNAs end up free in the systemic circulation is still under debate; one of the most accepted theories is that they are exported by cells via exosomes (Mittelbrunn et al., 2011). Importantly, the exosomes can also be internalized by other cells, and the information provided by the blood stream components could also be received through the form of functional miRNAs which may modulate the function of the receiving cell (Jackson, 2009; Sancho and Sanchez-Madrid, 2005).

We found it quite intriguing that miR-33b and miR-92b were downregulated in the plasma of patients with BC. Pathway enrichment analysis revealed that that many of the predicted miRNA targets were involved in critical pathways known to affect BC progression, including the tumor growth factor-beta signaling pathway. Furthermore, analysis of the potential binding targets for miR-92 and miR-33 predicted three potential binding sites for miR-92b in the CD69 3′UTR and one unique site for miR-33b in the CD96 3′UTR and CTLA-4. Importantly, the CD69 protein is expressed by activated T-cells, including natural killers (NK) cells, CD96 is expressed by NK cells and is important in NK cell adhesion to its targeted cells, whereas CTLA-4 is expressed primarily by activated T-cells and dendritic cells (Sancho and Sanchez-Madrid, 2005; Fuchs and Colonna, 2006; Laurent et al., 2010). Furthermore, miR-33 has recently been associated with the modulation of cholesterol metabolism and is expressed primarily by macrophages (Burnet, 1970). We therefore rationalized that subsets of adaptive or innate immunologic responses may be the plasma “carriers” of some miRNAs, such as miR-92b and miR-33b, that release them in tissues and ultimately into the systemic blood circulation as part of a homeostatic mechanism. In this scenario, infections, trauma, or even the onset of cancer may activate the immune responses, including subsets of T-cells associated with the downregulation of miR-92b and miR-33b and the upregulation of CD69 and CD96 or CTLA-4 expressed by T-regulatory NK cells or macrophages. This “immunosurveillance theory,” first proposed by Paul Ehrlich in the early 1900s and subsequently developed further by Thomas and Burnet in the 1970s, currently includes the concept of tumor immunoediting, which is thought to continue during tumor development (Ostrand-Rosenberg, 2008). Both innate and adaptive immunity are believed to be involved in tumor biology and they both can promote tumor progression as well as mediate tumor destruction (Horie et al., 2010; Bunt et al., 2007). Furthermore, miR-33 is an intronic miRNA that has recently been shown to be coordinately expressed and processed with the precursor mRNA in which it resides, the sterol regulatory element-binding protein genes found primarily in the liver and macrophages (Burnet, 1970), which are believed to be important mediators in all aspects of immunity. The macrophages are an exceptionally heterogonous population of cells, and like T-cells, they can contribute to tumor destruction or facilitate tumor growth and metastasis, depending of their phenotype (Horie et al., 2010). It is now accepted that “classically activated” macrophages via interferon-gamma function as activators of cytotoxic T-cells, whereas macrophages activated through the “alternate” pathway with interleukin-4, interleukin-13, or tumor growth factor-beta promote tumor progression by enhancing angiogenesis and producing type 2 cytokines and chemokines (Horie et al., 2010; Bunt et al., 2007). Furthermore, most progressively growing tumors are infiltrated by large numbers of macrophages. These tumor-associated macrophages are key components of the tumor stroma an essential component for the angiogenesis and matrix remodeling that support progressively growing neoplasms (Bunt et al., 2007). Interestingly, the pathway analysis of the most deregulated miRNAs in the plasma of individuals without BC and patients with controls revealed that, indeed, tumor growth factor-beta signaling pathway appeared to be heavily involved in this distinguishing the controls from MIBC cases.

The results indicate that plasma miRNAs-derived BC footprint are useful for predicting clinical outcome and that this footprint is a result of tumor-derived miRNAs and immune-cells-derived miRNAs reflecting the escape from tumor surveillance, in certain embodiments. Knowing the modulation and the exact source of these circulating (non-tumor-derived) miRNAs may be valuable for predicting clinical outcome, although their informative value in a cancer type-specific setting, such as BC, should be judged in conjunction with the tumor-derived miRNA footprint. The studies with the present invention showed that patients with MIBC displayed highly specific systemic miRNA profiles, which aids in distinguishing among other pathologies usually encountered in these age groups. This finding is of notable clinical consequence and is useful for affecting clinical practice patterns by directing the appropriate management of BC and thereby reducing death from BC. The employ of reliable markers also is useful to reduce the cost of health care delivery by improving and streamlining surveillance protocols and by personalizing therapy.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### PUBLICATIONS

Dinney C P, McConkey D J, Millikan R E, Wu X, Bar-Eli M, Adam L, et al. Focus on bladder cancer. Cancer Cell. 2004; 6(2):111-16.
Wu X R. Urothelial tumorigenesis: A tale of divergent pathways. Nat Rev Cancer 2005; 5:713-25.
Calin G A, Croce C M. MicroRNA signatures in human cancers. Nat Rev Cancer 2006; 6(11):857-66.
Bartel D P. MicroRNAs: target recognition and regulatory functions. Cell 2009; 23:136(2):215-33.
Cortez M A, Calin G A. MicroRNA Identification in plasma and serum. A new tool to diagnose and monitor diseases. Expert Opin Biol Ther 2009; 9(6):703-711.
Liu C G, Calin G A, Volinia S, Croce CM. MicroRNA expression profiling using microarrays. Nat Protoc 2008; 3(4):563-78.
Gentleman R C, Carey V J, Bates D M, Bolstad B, Dettling M, Dudoit S, et al. Bioconductor: Open software development for computational biology and bioinformatics. Genome Biology 2004; 5:R80.
Huber W, Von Heydebreck A, Sultmann H, Poustka A, Vingron M. Variance stabilization applied to microarray data calibration and to the quantification of differential expression. Bioinformatics 2002; 8(1):S96-104.
Gehlenborg N, O'Donoghue S I, Baliga N S, Grossmann A, Hibbs M A, Kitano H, et al. Visualization of omics data for systems biology. Nature Methods 2010; 7(3): S56-68.
Duda R O, Hart P E, Stork D G. Pattern classification. California: J Wiley & Sons; 2001.
Opgen R, Strimmer K. Accurate ranking of differentially expressed genes by a distribution-free shrinkage. Stat Appl Genet Mol Biol 2007; 6(Article9).
Strimmer K. A unified approach to false discovery rate estimation. BMC Bioinformatics 2008; 9:303.
Zhu J, Hastie T K. Logistic Regression and the Import Vector Machine. Journal of Computational and Graphical Statistics 14 2005; 1:185-20.
Zien A, Krämer N, Ratsch G, Sonnenburg S. The Feature Importance Ranking Measure. Machine Learning and Knowledge Discovery in Databases 2009; 694-709.
Catto J W, Miah S, Owen H C, Bryant H, Myers K, Dudziec E, et al. Distinct microRNA alterations characterize high- and low-grade bladder cancer. Cancer Res 2009; 69(21):8472-81.
Mittelbrunn M, Gutiérrez-Vázquez C, Villarroya-Beltri C, González S, Sánchez-Cabo F, González M A, et al. Unidirectional transfer of microRNA-loaded exosomes from T cells to antigen-presenting cells. Nat Commun 2011; 2:282.
Valadi H, Ekström K, Bossios A, Sjöstrand M, Lee J J, Lötvall J O. Exosome-mediated transfer of mRNAs and microRNAs is a novel mechanism of genetic exchange between cells. Nat Cell Biol 2007; 9(6):654-9.
Jackson D B. Serum-based microRNAs: Are we blinded by potential? Proc Natl Acad Sci USA 2009; 106(1).
Sancho D, Gómez M, Sánchez-Madrid F. CD69 is an immunoregulatory molecule induced following activation. Trends Immunol 2005; 26(3):136-40.
Fuchs A, Colonna M. The role of NK cell recognition of nectin and nectin-like proteins in tumor immunosurveillance. Semin Cancer Biol 2006; 16(5):359-66.
Laurent S, Carrega P, Saverino D, Piccioli P, Camoriano M, Morabito A, et al. CTLA-4 is expressed by human monocyte-derived dendritic cells and regulates their functions. Hum Immunol 2010; 71(10):934-41.
Horie T, Ono K, Horiguchi M, Nishi H, Nakamura T, Nagao K, et al. MicroRNA-33 encoded by an intron of sterol regulatory element-binding protein 2 (Srebp2) regulates HDL in vivo. Proc Natl Acad Sci USA. 2010; 107(40):17321-6.
Burnet, F M. The concept of immunological surveillance. Prog Exp Tumor Res 1970; 13:1-27.
Ostrand-Rosenberg S Immune surveillance a balance between protumor and antitumor immunity. Curr Opin Genet Dev 2008; 18:11-8.
Horie T, Ono K, Horiguchi M, Nishi H, Nakamura T, Nagao K, et al. MicroRNA-33 encoded by an intron of sterol regulatory element-binding protein 2 (Srebp2) regulates HDL in vivo. Proc Natl Acad Sci USA. 2010; 107(40):17321-6.
Bunt S K, Yang L, Sinha P, Clements V K, Leips J, Ostrand-Rosenberg S, et al. Reduced inflammation in the tumor microenvironment delays the accumulation of myeloid-derived suppressor cells and limits tumor progression. Cancer Res 2007; 67(20):10019-26.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for diagnosing bladder cancer or bladder cancer type in an individual, comprising the step of assaying expression of miRNA in a sample from the individual, wherein the miRNA is selected from the group consisting of the miRNAs of FIG. 2, the miRNAs of FIG. 4, and a combination thereof.

2. The method of claim 1, further defined as assaying expression of 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, 33 or more, 34 or more, 35 or more, 36 or more, 37 or more, 38 or more, or 39 or more miRNAs from the sample.

3. The method of claim 1, wherein the level of at least one miRNA in the sample is greater than the level of the corresponding miRNA in a normal sample or standard.

4. The method of claim 1, wherein the level of at least one miRNA in the sample is less than the level of the corresponding miRNA in a normal sample or standard.

5. The method of claim 1, wherein the expression of two or more miRNAs from the sample is compared to the expression of two or more miRNAs from a normal sample or standard.

6. The method of claim 1, further comprising:
(a) identifying the individual as having a bladder cancer if the expression level of one or more of hsa-miR-520c-3p-AS, hsa-miR-566-P; hsa-miR-33a-AS; hsa-miR-1254; hsa-miR-487a; hsa-miR-1273; hsa-miR-541; hsa-miR-487b; hsa-miR-148b; or hsa-miR-634 is increased in the sample relative to a reference or if the expression level of one or more of hsa-miR-92b; hsa-miR-1826; hsa-miR-92b*-AS; hsa-miR-33b; hsa-miR-1246; hsa-miR-1290; hsa-miR-1268-AS; hsa-miR-1914; hsa-miR-923-P; hsa-miR-23a; hsa-miR-923; hsa-miR-1469-AS; hsa-miR-184-P; hsa-miR-219-1-3p; hsa-miR-25; hsa-miR-935; hsa-miR-23b; hsa-miR-92a; hsa-miR-1228*-AS; hsa-miR-1181; hsa-miR-155*MM1T/C; hsa-miR-195*; hsa-miR-155MM1G/A; hsa-miR-1197; hsa-miR-548h; hsa-miR-32; hsa-miR-720; hsa-miR-202-AS or hsa-miR-937-AS is decreased in the sample relative to a reference; or
(b) identifying the individual as not having a bladder cancer if the expression level of hsa-miR-520c-3p-AS, hsa-miR-566-P; hsa-miR-33a-AS; hsa-miR-1254; hsa-miR-487a; hsa-miR-1273; hsa-miR-541; hsa-miR-487b; hsa-miR-148b; or hsa-miR-634 is not increased in the sample relative to a reference or if the expression level of hsa-miR-92b; hsa-miR-1826; hsa-miR-92b*-AS; hsa-miR-33b; hsa-miR-1246; hsa-miR-1290; hsa-miR-1268-AS; hsa-miR-1914; hsa-miR-923-P; hsa-miR-23a; hsa-miR-923; hsa-miR-1469-AS; hsa-miR-184-P; hsa-miR-219-1-3p; hsa-miR-25; hsa-miR-935; hsa-miR-23b; hsa-miR-92a; hsa-miR-1228*-AS; hsa-miR-1181; hsa-miR-155*MM1T/C; hsa-miR-195*; hsa-miR-155MM1G/A; hsa-miR-1197; hsa-miR-548h; hsa-miR-32; hsa-miR-720; hsa-miR-202-AS or hsa-miR-937-AS is not decreased in the sample relative to a reference.

7. The method of claim 6, wherein identifying the individual comprises reporting miRNA expression levels from the sample or reporting whether the individual has a bladder cancer.

8. The method of claim 7, wherein the reporting comprises providing a written or electronic report.

9. The method of claim 1, further comprising:
(a) identifying the individual as having an invasive bladder cancer if the expression level of one or more of hsa-miR-604; hsa-miR-940-P; hsa-miR-181a-2*-AS; hsa-miR-423-3p; hsa-miR-541; hsa-miR-522-AS; hsa-miR-574-3p; hsa-miR-1263-P; hsa-miR-338-3p-AS; hsa-miR-212; hsa-miR-200b; hsa-miR-671-3p; hsa-miR-1255p; hsa-miR-1262; hsa-miR-553; hsa-miR-544; hsa-miR-1248-P; hsa-miR-1233; hsa-miR-520c-3p-AS; or hsa-miR-520d-3p-AS is increased in the sample relative to a reference or if the expression level of one or more of hsa-miR-1826; hsa-miR-1246; hsa-miR-33b; hsa-miR-92b; hsa-miR-1290; hsa-miR-92a; hsa-miR-1268-AS; hsa-miR-1914; hsa-miR-92b*-AS; hsa-miR-219-1-3p; hsa-miR-25; hsa-miR-184-P; hsa-miR-1250-P; hsa-miR-302b; hsa-miR-373*-AS; hsa-miR-923-P; hsa-miR-923; hsa-miR-494; hsa-miR-1469-AS; or hsa-miR-23 is decreased in the sample relative to a reference; or
(b) identifying the individual as not having an invasive bladder cancer if the expression level of hsa-miR-604; hsa-miR-940-P; hsa-miR-181a-2*-AS; hsa-miR-423-3p; hsa-miR-541; hsa-miR-522-AS; hsa-miR-574-3p; hsa-miR-1263-P; hsa-miR-338-3p-AS; hsa-miR-212; hsa-miR-200b; hsa-miR-671-3p; hsa-miR-1255p; hsa-miR-1262; hsa-miR-553; hsa-miR-544; hsa-miR-1248-P; hsa-miR-1233; hsa-miR-520c-3p-AS; or hsa-miR-520d-3p-AS is not increased in the sample relative to a reference or if the expression level of hsa-miR-1826; hsa-miR-1246; hsa-miR-33b; hsa-miR-92b; hsa-miR-1290; hsa-miR-92a; hsa-miR-1268-AS; hsa-miR-1914; hsa-miR-92b*-AS; hsa-miR-219-1-3p; hsa-miR-25; hsa-miR-184-P; hsa-miR-1250-P; hsa-miR-302b; hsa-miR-373*-AS; hsa-miR-923-P; hsa-miR-923; hsa-miR-494; hsa-miR-1469-AS; or hsa-miR-23 is not decreased in the sample relative to a reference.

10. The method of claim 1, wherein differential expression of two or more miRNAs from the sample compared to a normal sample or standard identifies the presence or type of bladder cancer in the individual.

11. The method of claim 10, wherein the bladder cancer is muscle-invasive bladder cancer or non-muscle-invasive bladder cancer.

12. The method of claim 1, wherein the sample is selected from the group consisting of blood, plasma, serum, urine, biopsy, and semen.

13. The method of claim 1, further comprising the step of analyzing a sample from the individual using an additional method for diagnosing bladder cancer.

14. The method of claim 10, wherein the additional method for diagnosing bladder cancer is selected from the group consisting of medical interview, physical examination, urinalysis, urine cytology, cystoscopy, ultrasound, pyelography, CT scan, and a combination thereof.

15. The method of claim 1, further comprising the step of obtaining the sample from the individual.

16. The method of claim 1, wherein the individual has at least one symptom selected from the group consisting of blood in the urine, pain or burning during urination without evidence of urinary tract infection, having to urinate more often, and feeling the strong urge to urinate without producing much urine.

17. The method of claim 1, wherein the individual has a personal or family history of bladder cancer.

18. The method of claim 1, wherein the individual is asymptomatic or is undergoing routine medical testing.

19. The method of claim 1, wherein the assaying identifies the stage of the bladder cancer.

20. The method of claim 19, wherein the stage of bladder cancer is stage CIS, Tₐ, T₁, T₂, T₃, T₄, or T₁₋₄N₁₋₂M₁₋₂.

21. An array comprising miRNA probes that are complementary to one or more of the miRNAs selected from the group consisting of the miRNAs of FIG. 2 and FIG. 4, wherein said miRNA probes are immobilized on a solid support.

22. The array of claim 21, wherein 50%, 55%, 60%, 65%, 70%, 75%, 80%, 85%, 90%, 95%, 97%, 98%, 99%, or 100% of the miRNA probes on the array are complementary to one or more of the miRNAs selected from the group consisting of the miRNAs of FIG. 2 and FIG. 4.

23. A kit for diagnosing bladder cancer, comprising:
a) the array of claim 21; and/or
b) one or more miRNA probes that are complementary to a miRNA selected from the group consisting of the miRNAs of FIG. 2 and FIG. 4, wherein the items in the kit are housed in a suitable container.

24. A method of treating an individual diagnosed with a bladder cancer by a method of claim 1 comprising administering an anticancer therapy to the individual.

25. The method of claim 24, wherein the anticancer therapy is a chemotherapy, radiotherapy, gene therapy, surgery, hormonal therapy, anti-angiogenic therapy or cytokine therapy.

26. The method of claim 24, wherein the individual is diagnosed with muscle-invasive bladder cancer.
